(19) United States
(12) Patent Application Publication
(10) Pub. No.: US 2008/0173300 A1
(43) Pub. Date: Jul. 24, 2008

(54) PORTABLE VAPORIZED SUBSTANCE DELIVERY SYSTEM

(76) Inventor: Michael Justman, Calabasas, CA (US)

Correspondence Address: TROJAN LAW OFFICES, 9250 WILSHIRE BLVD, SUITE 325, BEVERLY HILLS, CA, 90212, US

(21) Appl. No.: 11/626,813

(22) Filed: Jan. 24, 2007

(51) Int. Cl. A61M 15/00

(52) U.S. Cl. 128/200.11

(57) ABSTRACT

A portable device for delivering inhalable vapors comprises a portable hand-held refillable vaporization chamber with an inlet for outside air and an outlet for inhaling vapors. The chamber can be heated by a small heat source, such as a cigarette lighter, so that the contents become vaporized almost instantly. Optionally, the chamber is connected to a storage reservoir.

### BACKGROUND OF THE INVENTION

Many therapeutic substances can be inhaled. In fact, it is often preferable to inhale a medication rather than to take a pill. For example, cold medications are effective at relieving sinus congestion when inhaled directly through the nose. The same is true for sore throat and asthma medications that can both be effective immediately if inhaled. Because inhaling involves targeted delivery, a lot less medication can be used to achieve the same result. As with therapeutic substances, alcohol can also be inhaled. One can get the “desired” level of alcoholic intoxication from simply inhaling alcohol vapors instead of drinking. A recently introduced party gadget called AWOL or “Alcohol Without Liquid” takes advantage of this fact. AWOL has a beneficial side: inhalers end up with much less alcohol in their system than drinkers with the same desired effect.

Unfortunately, creating and inhaling vapors is not easy to do on the go. The therapeutic substance needs to be heated or otherwise vaporized (converted from solid or liquid phase into the gas phase). Usually one has to carry around a container of preheated liquid, such as a thermos flask or a thermal mug. For example, a person with a cold may leave home with a large container of herbal tea and sip it or inhale the vapors during the commute. To have the hot liquid available for longer times, one has to carry around a larger container, which is inconvenient. Such a person would benefit from being able to quickly generate small dozes of inhalable therapeutic vapors throughout the day without the need to carry a large flask.

A simple method of generating therapeutic vapors is described in the U.S. Pat. No. 6,295,982. The patent teaches placing a large tablet on the upper lip of the user. The heat from the skin causes the contents of the tablet to evaporate directly into the nose. The same principle underlies the popular VAPORUB® product sold under the VICKS® brand. This approach, while convenient, is limited by the volatility of the therapeutic substance. Specifically, the substance must evaporate at the surface temperature of the skin (approximately 37° C). Water-based solutions, such as herbal teas and extracts are not volatile enough to be used in this approach.

Actual steam or vapor for inhaling is produced by vaporizer devices. Vaporizers have many advantages over smoking devices. Most importantly, vaporizers convert the desired compound into gas without burning. Thus the user is protected from the negative effects of carbon monoxide, tar and other compounds found in smoke. At the same time, a vaporized substance is heated to a lower temperature lower than a burning substance and does not undergo thermal degradation.

Vaporizers are used for various compounds, ranging from plain water and alcohol to medicinal herbs and surgical anesthetics. A basic example of a vaporizer is a humidifier: a device producing steam or water vapor. Such a device includes a container for holding water, a heater for turning the water into steam and an outlet for the steam. The steam can be inhaled directly or released into the environment and humidify the air in the room (U.S. Pat. No. 5,611,967). Alternatively, a humidifier can be connected to an assisted breathing apparatus and humidify the air delivered to the patient's lungs (U.S. Pat. No. 4,355,636). Other commonly used humidifiers are “home spas” manufactured for example, by Homedica Co. These devices have a nozzle for enclosing the user's face, so that the entire face is bathed in the emanating steam. The steam may include water or a therapeutic solution beneficial to the facial skin or the upper respiratory tract. Unfortunately, such devices are fairly large. Some also use electricity to power the heater. As a result, the devices are limited to home use.

Portable inhalers are also known in the art. Inhaling various substances with the use of water has been a part of traditional practices for centuries. For example, water pipes are used in different regions of the world and known as hookahs, neghrils or bongs. Such devices involve burning solid material, for example dry herb or fruit, and allow the smoke to pass through water for filtration. The filtered smoke is less harmful than the smoke inhaled directly.

Like stationary humidifiers and home spas, water pipes have several limitations. The first one is limited portability. While they can be easily moved, water pipes are too large to easily carry around the entire day. Besides the size, the water pipes are unsafe to carry while the herb is burning. In addition to portability, a more serious limitation is the fact that liquid medicinal compounds may not be used with a water pipe. Only solid materials capable of burning and generating smoke can be inhaled through a hookah. Most importantly, however, hookahs are generating smoke and not steam.

Truly portable inhalers are also known in the art. One group of such inhalers involves vapor-holding chambers. Once filled with vapor, the chamber can be carried around and used until the vapor has been exhausted. (See for example, U.S. Pat. Nos. 5,309,900 and 6,718,973). These devices are much more safe and effective than a bowl or mug of heated liquid that one would carry around in order to inhale the vapor. However, the devices require constant refill from a stationary evaporator. The “AWOL” or Alcohol Without Liquid party device mentioned above also has this problem. The individual portable device needs to be constantly refilled from a charging unit that converts liquid alcohol into vapor.

Another example of a portable vaporizer is a nebulizer or an atomizer. Such a device turns solid or liquid substance into aerosol using air, oxygen or a propellant gas. The aerosol can then be inhaled and instantly delivered to the lungs. The most common nebulizer is an “asthma inhaler” or “allergy inhaler” that delivers medication directly to the bronchi to alleviate the constriction caused by an allergic reaction or an asthma attack. Because they do not require heating units, nebulizers are often small, portable hand-held devices. However, nebulizers cannot be manually refilled. As a result, they are either single-use or refillable using stationary pumps. Most often, nebulizers come pre-filled with a particular medication and are discarded after use.

In summary, there are no examples of a truly portable inhaler that would have the convenience of an asthma inhaler, but be refillable and adaptable to a variety of substances. A user would be able to take such a device anywhere and quickly and easily prepare and inhale the substance of choice.

### SUMMARY OF THE INVENTION

The present invention is a portable system for delivering inhalable substances in a vaporized form. The system comprises a hand-held refillable vaporization chamber having two outlets: one for venting, and the other for inhaling vapors. The chamber is small enough so that its contents can be heated and vaporized almost instantly by a small heat source, such a cigarette lighter or a candle. Optionally, the chamber is connected to a refill storage reservoir.

### BRIEF DESCRIPTION OF THE DRAWINGS

FIGS. 1A through 1F show the first preferred embodiment of the vaporizer.

FIGS. 2A and 2B show the second preferred embodiment of the vaporizer with a storage reservoir attached.

FIG. 3 shows the use of the vaporizer.

### DETAILED DESCRIPTION OF THE INVENTION

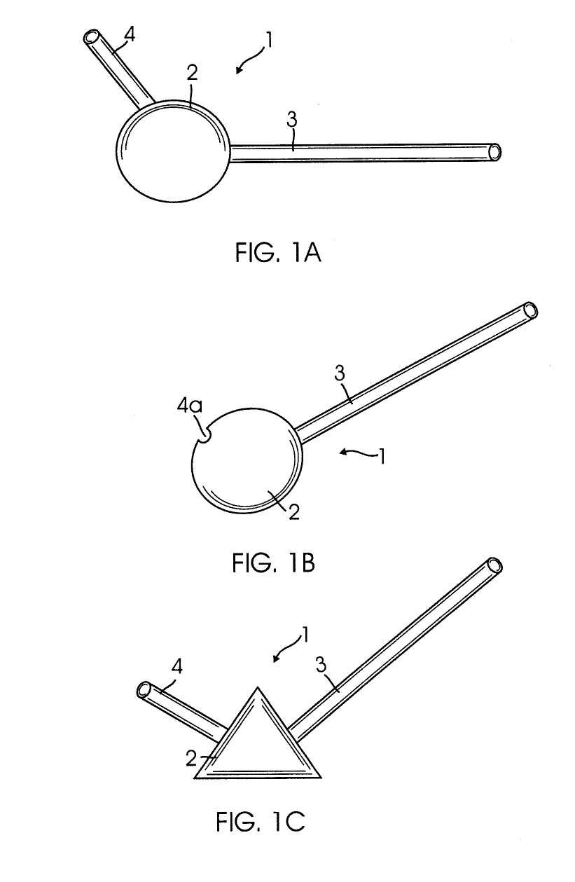

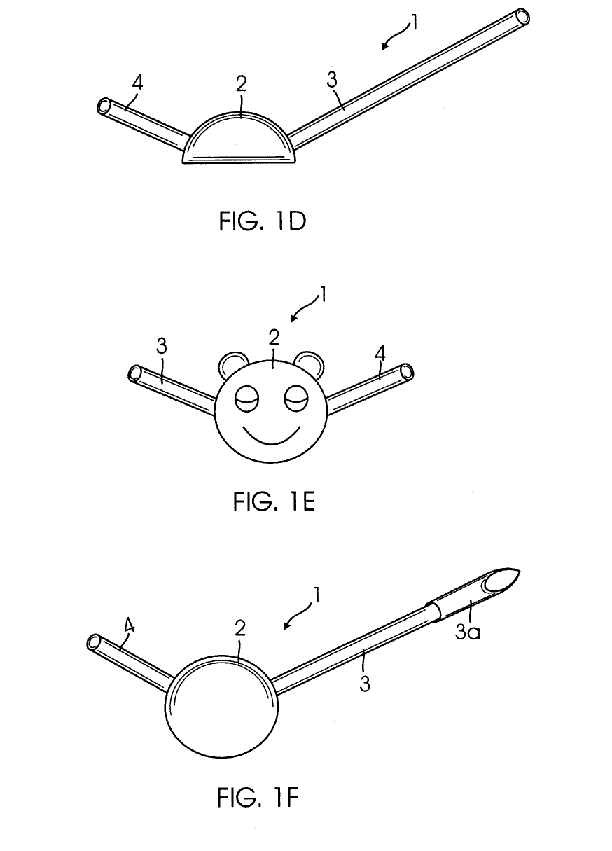

The present invention is a portable vaporized compound delivery system consisting of a hand-held vaporizer. As shown on FIG. 1A, the vaporizer 1 has a liquid chamber 2 with two openings and two tubes (or vapor pathways) 3 and 4 emanating therefrom. As shown on FIG. 1B, the second tube 4 emanating from the opening 4a is optional. The vaporization chamber may take any shape. The container may also have a decorative shape, for example that of a toy (FIG. 1E) or a cartoon character in order to appeal to children and encourage their compliance with the treatment regimen.

The chamber may be made of any heat-resistant material. The material may be hard or flexible. Examples of the hard material include glass, ceramic and heat-resistant plastic. The main advantage of the hard chamber is its ability to retain shape. Such a container may be placed on a surface and used hands-free. To facilitate such placing, the container may have a flat bottom surface. The geometric shape of such a container may be a rectangle, a pyramid (FIG. 1C) or a sphere with a flattened out portion (FIG. 1D) or any other flat-bottomed shape.

The examples of heat-resistant flexible material include a variety of heat resistant polymers and other plastics. The main advantage of a flexible material is its ability to collapse when empty. Such property allows the user to stow away the vaporizer more easily.

The chamber can be heated by a small heat source, such as a cigarette lighter or a candle, so that the contents become vaporized almost instantly. The size of the chamber must be small so that the heat capacity permits such a small heat source to cause evaporation of the liquid. Preferably the chamber is about one inch to several inches in diameter or cross-section.

The tubes 3 and 4 can have identical sizes, or differ in length or diameter or both. As shown on FIG. 1B, the second tube may be absent. The tubes can be made of glass, plastic or any other suitable heat-resistant material. The material can be either rigid of flexible. Each option has its advantages: a rigid tube can be used hands-free and hold steady, while a flexible tube may allow movement during use. In addition, during storage, the flexible tube may be safely folded or wrapped without breaking. The tubes can be permanently fused to the chamber, as with glass or moldable plastic. Alternatively, the tubes may be screwed on or attached with snap-on seals or any other seals known in the art. Having the detachable tubes can facilitate the cleaning of the vaporizer.

For convenience and hygiene, the inhaling tube may come with a replaceable, disposable nozzle, as shown in FIG. 1F.

To ensure that the device if filled with liquid, is also portable, the device may be supplemented with corks or plugs to close the openings or tubes attached thereto.

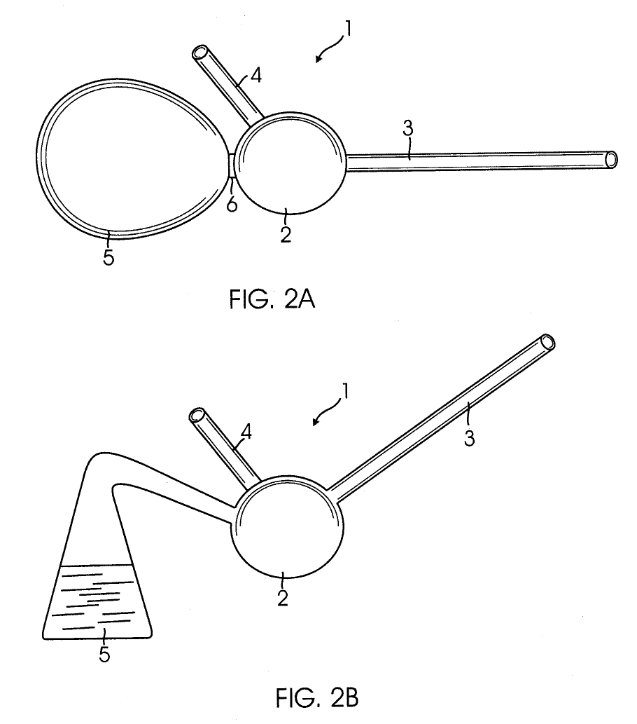

In the second preferred embodiment, shown on FIGS. 2A and 2B, the vaporizer has a storage chamber 5 connected to the vaporization chamber 2. The storage chamber may be similar in size or bigger than the vaporization chamber. The storage chamber may be made of the same material as the vaporization chamber or a different material. The storage chamber may be separated from the vaporization chamber by any resealable means 6 known in the art. Alternatively, the storage chamber may be connected without any seal. If the storage chamber 5 is made of a rigid material, it is positioned such that extra liquid does not spill into the evaporation chamber unless intended (FIG. 2B).

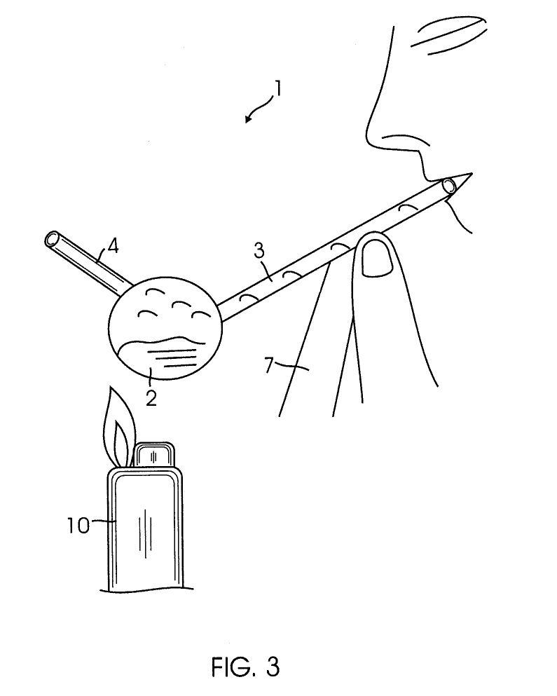

The operation of the vaporizer is shown on FIG. 3. For convenience, the vaporizer is depicted such that the liquid inside is visible through the walls. The container 2 is filled with liquid. Heating is accomplished by a cigarette lighter 10, sufficient to heat up the contents of the container 2. The user's fingers 7 are holding the device. The device can be held by either tube 3 or 4. Vapor is inhaled through the tube 3 by the user's nose or mouth. To allow unrestricted inhalation, outside air is let in through the other tube 4. The outside air mixes with the vapor in the chamber. The inhalation may continue until all the liquid has evaporated from the chamber 2. If inhalation is no longer desired, the vapors may be allowed to escape through both tubes, 3 and 4 with the help of continued heating. Alternatively, the leftover liquid may be simply poured out. The latter is especially convenient if the tubes 3 and 4 are detachable.

While the invention has been described in detail with reference to specific embodiments, it will be apparent to one skilled in the art that various modifications can be made within the scope of this invention. Thus the scope of the invention should not be limited by any of the examples described herein but by the claims presented below.

I claim:

1. A vaporized liquid delivery system comprising:
a chamber for vaporizing a liquid, said chamber having two openings;
at least one vapor pathway connected to at least one of said openings;
wherein said chamber may be heated by an external heat source to achieve vaporization of said liquid.

2. The vaporized liquid delivery system of claim 1 wherein said liquid has therapeutic properties.

3. The vaporized liquid delivery system of claim 1 wherein said liquid is selected from a group consisting of water, alcohol, water-based solution, alcohol-based solution and herbal extract.

4. The vaporized liquid delivery system of claim 1 wherein said chamber is between 1 inch and 3 inches in diameter.

5. The vaporized liquid delivery system of claim 1 wherein said chamber is collapsible.

6. The vaporized liquid delivery system of claim 1 wherein said vapor pathways are bendable.

7. The vaporized liquid delivery system of claim 1 further comprising a disposable attachment capable of being connected to at least one of said vapor pathways and further capable of being inserted into the user's mouth.

8. The vaporized liquid delivery system of claim 1 further comprising closing means to close said openings or said vapor pathways connected thereto.

9. A vaporized liquid delivery system comprising:
a first chamber for vaporizing a liquid, said chamber having two openings;
at least one vapor pathway connected to at least one of said openings;
wherein said chamber may be heated by an external heat source to achieve vaporization of said liquid;
a second chamber connected to said first chamber, wherein said second chamber may hold extra amount of said liquid; and
means for segregating the liquids in said first and said second chambers.
